(19) United States
(12) Patent Application Publication
(10) Pub. No.: US 2011/0065674 A1
(43) Pub. Date: Mar. 17, 2011

(54) METHODS AND COMPOSITIONS FOR IMPROVING COGNITIVE FUNCTION

(75) Inventors: Michela Gallagher, Baltimore, MD (US); Ming Teng Koh, Baltimore, MD (US)

(73) Assignee: THE JOHNS HOPKINS UNIVERSITY

(21) Appl. No.: 12/992,379

(22) PCT Filed: May 14, 2009
(86) PCT No.: PCT/US09/03030

§ 371 Date: Nov. 12, 2010

Related U.S. Application Data
(60) Provisional application No. 61/127,534, filed on May 14, 2008.

(51) Int. Cl. A61K 31/445; A61K 31/473; A61K 31/27; A61K 31/407; A61K 31/662; A61P 25/28; A61P 25/00

(52) U.S. Cl. 514/129; 514/319; 514/297; 514/490; 514/411

(57) ABSTRACT

The invention relates to methods and compositions for improving cognitive function by using a combination of valproic acid and analogs and derivatives thereof (“valproate”) and an acetylcholinesterase inhibitor (AChEI). In particular, it relates to the use of a combination of a valproate and an AChEI in treating age-related cognitive impairment in a subject in need or at risk thereof, including, without limitation, subjects having or at risk for Mild Cognitive Impairment (MCI), Age-Associated Memory Impairment (AAMI) or Age-Related Cognitive Decline (ARCD).

### FIELD OF THE INVENTION

The invention relates to methods and compositions for improving cognitive function by using a combination of valproic acid and analogs and derivatives thereof (“valproate”) and an acetylcholinesterase inhibitor (AChEI). In particular, it relates to the use of a combination of a valproate and an AChEI in treating age-related cognitive impairment in a subject in need or at risk thereof, including, without limitation, subjects having or at risk for Mild Cognitive Impairment (MCI), Age-Associated Memory Impairment (AAMI) or Age-Related Cognitive Decline (ARCD).

### BACKGROUND OF THE INVENTION

Cognitive and/or degenerative brain disorders are characterized clinically by progressive loss of memory, cognition, reasoning, judgment and emotional stability that gradually leads to profound mental deterioration and ultimately death. Among these diseases, Alzheimer's disease is considered as the most common and is believed to represent the fourth most common medical cause of death in the United States. In 1997, Alzheimer's disease was estimated to affect more than 2 million people in the United States, a number expected to quadruple within the next 50 years (Brookmeyer et al., 1998).

Cognitive ability may decline as a normal consequence of aging. A significant population of elderly adults experiences a decline in cognitive ability that exceeds what is typical in normal aging. Such age-related loss of cognitive function is characterized clinically by progressive loss of memory, cognition, reasoning, and judgment. Mild Cognitive Impairment (MCI), Age-Associated Memory Impairment (AAMI), Age-Related Cognitive Decline (ARCD) or similar clinical groupings are among those related to such age-related loss of cognitive function.

According to some estimates, MCI affects 5-5.7 million patients in the US over the age of 65 (Yesavage et al., 2002; Hanninen et al., 2002; Plassman et al., 2008). Additionally, the number of patients falling in the category of Age-Associated Memory Impairment or similar diagnostic categories (e.g., Age-Related Cognitive Decline) is also staggering. For example, according to the estimates of Barker et al. (1995) there are more than 16 million people with Age-Associated Memory Impairment in the U.S. alone.

There is, therefore, a need for effective treatment for age-related cognitive impairment and to improve cognitive function in patients diagnosed with MCI, AAMI, ARCD and similar age-associated cognitive impairments or at risk of developing them.

Tacrine hydrochloride (“COGNEXT™”), the first FDA approved drug for Alzheimer's disease is an AChEI (Cutler et al, 1993). Other examples of clinically used AChEIs include galantamine (“REMINYL™”) or rivastigmine (“EXELON™”). These drugs, however, have shown limited success in cognitive improvement in Alzheimer's disease patients and display a use-limiting side effect profile. Another AChEI, donepezil (also known as “ARICEPT™”) appears more effective than tacrine. With donepezil, Alzheimer's disease patients show slight cognitive improvements (Barner et al, 1998; Rogers et al, 1998), but the usefulness of donepezil is also limited due to its moderate efficacy and side effects. The long-term therapeutic efficacy of donepezil has also been increasingly questioned (Maggini et al., 2006; Petersen et al., 2006). There is, therefore, still a need for more effective treatment for disorders involving cognitive dysfunction and in particular age-related cognitive impairment.

### SUMMARY OF THE INVENTION

In accordance with a first aspect of the present invention, there is provided a method for treating, or improving cognitive function in, a subject suffering from age-related cognitive impairment or at risk thereof, the method comprising the step of administering to said subject an AChEI or a pharmaceutically acceptable salt thereof in combination with valproic acid or an analog or a derivative thereof (together “valproate”) or a pharmaceutically acceptable salt thereof.

In certain embodiments of the invention, the age-related cognitive impairment is selected from the group consisting of AAMI, ARCD and MCI. In some embodiments, the age-related cognitive impairment is MCI.

In certain embodiments of the invention, the AChEI used in the methods of the invention is selected from the group consisting of donepezil, tacrine, rivastigmine, physostigmine, galanthamine, or metrifonate or acceptable pharmaceutically acceptable salts thereof. In some embodiments, the AChEI is donepezil or a pharmaceutically acceptable salt thereof.

In certain embodiments of the invention, the AChEI or a pharmaceutically acceptable salt thereof is administered every 12 or 24 hours at a daily dose of about 0.1 to 10 mg. In some embodiments, a subtherapeutic amount of the AChEI or pharmaceutically acceptable salts thereof is administered. In some embodiments, the subtherapeutic amount of the AChEI or its salt administered is a daily dose of less than about 10 mg, less than about 5 mg, less than about 2 mg, less than about 1 mg, less than about 0.5 mg, or less than about 0.1 mg.

In certain embodiments of the invention, the valproate used in the methods of the invention is valproic acid (2-propylpentanoic acid) or an analog or a derivative thereof, or pharmaceutically acceptable salts thereof. In some embodiments, the valproate is selected from a group consisting of 2-methylpent-2-enoic acid, or 2-methyl-2-n-propylpentanoic acid, or pent-4-enoic acid, or 2-ethylhexanoic acid, or valpromide, 2-propylpentanamide, 2-n-alkylbut-3-ynoic acid and their pharmaceutically acceptable salts, as well as any combinations thereof (see Kostrouchova et al, 2007). In certain embodiments of the invention, the valproate is sodium valproate. In certain embodiments of the invention, the valproate is hydrogen bis(2-propylpentanoate).

In certain embodiments of the invention, the valproate or a pharmaceutically acceptable salt thereof is administered at a daily dose such that the subject maintains a blood total valproate level of 0.1 to 20 μg/ml plasma. In some embodiments, a subtherapeutic amount of the valproate or pharmaceutically acceptable salts thereof is administered. In some embodiments, the subtherapeutic amount of the valproate administered is a daily dose of less than 1500 mg, less than 1000 mg, less than 500 mg, less than 250 mg, less than 125 mg, less than 75 mg, less than 25 mg, less than 10 mg, or less than 5 mg.

In certain embodiments of the invention, the AChEI and the valproate, or their pharmaceutically acceptable salts, are administered simultaneously, sequentially, or as a single formulation.

In accordance with a second aspect of the present invention, there is provided a pharmaceutical composition for treating, or improving cognitive function in, a subject suffering from age-related cognitive impairment or at risk thereof, the composition comprising an AChEI and a valproate or their pharmaceutically acceptable salts. In some embodiments, the composition is in a solid form. In some embodiments, the composition is in a liquid form. In some embodiments, the composition is in an aqueous solution. In some embodiments, the composition is in a unit dosage form.

In certain embodiments of the invention, the valproate in the composition is valproic acid (2-propylpentanoic acid) or an analog or a derivative thereof, or pharmaceutically acceptable salts thereof. In some embodiments, the valproate is selected from a group consisting of 2-methylpent-2-enoic acid, or 2-methyl-2-n-propylpentanoic acid, or pent-4-enoic acid, or 2-ethylhexanoic acid, or valpromide, 2-propylpentanamide, 2-n-alkylbut-3-ynoic acid and their pharmaceutically acceptable salts, as well as any combinations thereof (see Kostrouchova et al, 2007). In certain embodiments of the invention, the valproate is sodium valproate. In certain embodiments of the invention, the valproate is hydrogen bis(2-propylpentanoate). In some embodiments, the amount of the valproate or its salt is a dosage such that the subject maintains a blood total valproate level of 0.1 to 20 μg/ml plasma. In some embodiments, the amount of the valproate or its salt is a dose of less than 1500 mg, less than 1000 mg, less than 500 mg, less than 250 mg, less than 125 mg, less than 75 mg, less than 25 mg, less than 10 mg, or less than 5 mg. In some embodiments, the AChEI in the composition is selected from the groups consisting on donepezil, tacrine, rivastigmine, physostigmine, galanthamine, metrifonate and their salts. In some embodiments, the AChEI is donepezil or its salt. In some embodiments, the amount of the AChEI or its salt in the composition is about 0.1 to 10 mg. In some embodiments, the amount of the AChEI or its salt in the composition is less than about 10 mg, less than about 5 mg, less than about 2 mg, less than 1 mg, less than 0.5 mg, or less than 0.1 mg.

### BRIEF DESCRIPTION OF THE DRAWINGS

FIG. 1 Chronic valproate treatment increases muscarinic receptor-GTP-Eu binding in the hippocampus of memory impaired aged rats. YNG=young; AI-VEH=aged impaired vehicle treated; AI-VPA=aged impaired valproate treated.

FIG. 2 Memory-impaired aged rats treated with sodium valproate (“VPA”) or saline (“VEH”) performed at a comparable level at the end of the training phase, but those treated with a higher dose of VPA (VPA 100=100 mg/kg/day) showed less forgetting (i.e., shorter path length; see Retention; *=p<0.5) after a 6-hr delay compared to their counterparts treated with either saline or a lower dose of sodium valproate (VPA 50=50 mg/kg/day).

FIG. 3 depicts the effects of administering a combination of donezepil and sodium valproate (“VPA”) on the spatial memory retention of aged-impaired rats (AI) in a Morris Water Maze (MWM) test. A range of treatment conditions were employed: vehicle control, donepezil (0.5, 1 or 2 mg/kg) and donepezil (0.5, 1 or 2 mg/kg) combined with 100 mg/kg/day VPA. The AI rats were trained for two consecutive days, with a one-time treatment of donepezil (or vehicle) prior to the training trials per day. 24 hours later, the AI rats were tested in a memory retention trial. The time the AI rats spent swimming in the target quadrant or the target annulus in the memory retention trial is used as a measure of spatial memory retention. The target quadrant refers to the quadrant of the maze (which is a circular pool) where the escape platform is placed during the training trials. The target annulus refers to the exact location of the escape platform during the training trials. For each bar, the middle line represents the median, the top line represents the top quartile, and the bottom line represents the bottom quartile (*=p<0.019; #=p<0.030, Wilcoxon matched pairs test, 1 tail).

### DETAILED DESCRIPTION OF THE INVENTION

Unless otherwise defined herein, scientific and technical terms used in this application shall have the meanings that are commonly understood by those of ordinary skill in the art. Generally, nomenclature used in connection with, and techniques of, cell and tissue culture, molecular biology, cell and cancer biology, neurobiology, neurochemistry, virology, immunology, microbiology, pharmacology, genetics and protein and nucleic acid chemistry, described herein, are those well known and commonly used in the art.

The methods and techniques of the present invention are generally performed, unless otherwise indicated, according to conventional methods well known in the art and as described in various general and more specific references that are cited and discussed throughout this specification. See, e.g. “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); Gilbert et al., “Developmental Biology, 6th ed.”, Sinauer Associates, Inc., Sunderland, Mass. (2000).

Chemistry terms used herein are used according to conventional usage in the art, as exemplified by “The McGraw-Hill Dictionary of Chemical Terms”, Parker S., Ed., McGraw-Hill, San Francisco, Calif. (1985).

All of the above, and any other publications, patents and published patent applications referred to in this application are specifically incorporated by reference herein. In case of conflict, the present specification, including its specific definitions, will control.

Throughout this specification, the word “comprise” or variations such as “comprises” or “comprising” will be understood to imply the inclusion of a stated integer (or components) or group of integers (or components), but not the exclusion of any other integer (or components) or group of integers (or components).

The singular forms “a,” “an,” and “the” include the plurals unless the context clearly dictates otherwise.

The term “including” is used to mean “including but not limited to”. “Including” and “including but not limited to” are used interchangeably.

The term “agent” is used herein to denote a chemical compound (such as an organic or inorganic compound, a mixture of chemical compounds), a biological macromolecule (such as a nucleic acid, an antibody, including parts thereof as well as humanized, chimeric and human antibodies and monoclonal antibodies, a protein or portion thereof, e.g., a peptide, a lipid, a carbohydrate), or an extract made from biological materials such as bacteria, plants, fungi, or animal (particularly mammalian) cells or tissues. Agents include, for example, agents which are known with respect to structure, and those which are not known with respect to structure.

A “patient”, “subject”, or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

“Promoting” cognitive function refers to affecting age-related impaired cognitive function so that it more closely resembles the function of an aged-matched normal, unimpaired subject, or the function of a young adult subject. Cognitive function may be promoted to any detectable degree, but in humans preferably is promoted sufficiently to allow an impaired subject to carry out daily activities of normal life at the same level of proficiency as an aged-matched normal, unimpaired subject or as a young adult subject.

“Preserving” cognitive function refers to affecting normal or impaired cognitive function such that it does not decline or does not fall below that observed in the subject upon first presentation or diagnosis, or delays such decline.

“Improving” cognitive function includes promoting cognitive function and/or preserving cognitive function in a subject.

“Treating” a condition or patient refers to taking steps to obtain beneficial or desired results, including clinical results. Beneficial or desired clinical results include, but are not limited to, alleviation or amelioration of one or more symptoms associated with age-related cognitive impairment, delay or slowing of that impairment, amelioration, palliation or stabilization of that impairment, improvement of cognitive function or a reduced rate of decline of cognitive function in subjects with age-related cognitive impairment or at risk thereof.

“Administering” or “administration of a substance, a compound or an agent to a subject can be carried out using one of a variety of methods known to those skilled in the art. For example, a compound or an agent can be administered, intravenously, arterially, intradermally, intramuscularly, intraperitonealy, intravenously, subcutaneously, ocularly, sublingually, orally (by ingestion), intranasally (by inhalation), intraspinally, intracerebrally, and transdermally (by absorbtion, e.g., through a skin duct). A compound or agent can also appropriately be introduced by rechargable or biodegradable polymeric devices or other devices, e.g., patches and pumps, or formulations, which provide for the extended, slow or controlled release of the compound or agent. Administering can also be performed, for example, once, a plurality of times, and/or over one or more extended periods. In some aspects, the administration includes both direct administration, including self-administration, and indirect administration, including the act of prescribing a drug. For example, as used herein, a physician who instructs a patient to self-administer a drug, or to have the drug administered by another and/or who provides a patient with a prescription for a drug is administering the drug to the patient.

Appropriate methods of administering a substance, a compound or an agent to a subject will also depend, for example, on the age of the subject, whether the subject is active or inactive at the time of administering, whether the subject is cognitively impaired at the time of administering, the extent of the impairment, and the chemical and biological properties of the compound or agent (e.g. solubility, digestibility, bioavailability, stability and toxicity). Preferably, a compound or an agent is administered orally, e.g., to a subject by ingestion. In some embodiments, the orally administered compound or agent is in an extended release or slow release formulation, or administered using a device for such slow or extended release.

As used herein, administration of a valproate and an AChEI or pharmaceutically acceptable salts thereof “in combination” includes simultaneous administration and/or administration at different times, such as sequential administration.

The term “simultaneous administration,” as used herein, means that the valproate and the AChEI, or their pharmaceutically acceptable salts, are administered with a time separation of no more than about 15 minutes, and preferably no more than about 10 minutes. When the drugs are administered simultaneously, the valproate and the AChEI, or their salts, may be contained in the same dosage (e.g., a unit dosage form comprising both the valproate and AChEI, or their salts) or in discrete dosages (e.g., the valproate or its salt is contained in one dosage form and the AChEI or its salt is contained in another dosage form).

The term “sequential administration” as used herein means that the valproate and the AChEI, or their pharmaceutically acceptable salts, are administered with a time separation of more than about 15 minutes, and preferably more than about one hour, or up to 12 hours. Either valproate or its salt or the AChEI or its salt, may be administered first. The valproate and the AChEI, or their salts, for sequential administration may be contained in discrete dosage forms, optionally contained in the same container or package.

A “therapeutically effective amount” of a drug or agent is an amount of a drug or an agent that, when administered to a subject will have the intended therapeutic effect, e.g. treating, or improving cognitive function in, a subject, e.g., a patient with age-related cognitive impairment or a patient at risk thereof. The full therapeutic effect does not necessarily occur by administration of one dose, and may occur only after administration of a series of doses. Thus, a therapeutically effective amount of an agent or compound this invention may be administered in one or more administrations. The precise effective amount needed for a subject will depend upon, for example, the subject's size, health and age, the nature and extent of the cognitive impairment, and the therapeutics or combination of therapeutics selected for administration, and the mode of administration. The skilled worker can readily determine the effective amount for a given situation by routine experimentation.

“Subtherapeutic amount” refers to an amount administered of an agent or compound of the invention that is less than the therapeutic amount, that is, less than the amount normally used when said agent or compound is administered alone (i.e., individually and not in combination with other therapeutic agents or compounds) to treat disorders involving cognitive dysfunction.

“Pharmaceutically acceptable salts” is used herein to refer to an agent or a compound according to the invention that is a therapeutically active, non-toxic base and acid salt form of the agents compounds of the invention.

“Cognitive function” or “cognitive status” refers to any higher order intellectual brain process or brain state, respectively, involved in learning and/or memory including, but not limited to, attention, information acquisition, information processing, working memory, short-term memory, long-term memory, anterograde memory, retrograde memory, memory retrieval, discrimination learning, decision-making, inhibitory response control, attentional set-shifting, delayed reinforcement learning, reversal learning, the temporal integration of voluntary behavior, and expressing an interest in one's surroundings and self-care.

In humans, cognitive function may be measured, for example and without limitation, by the Alzheimer's Disease Assessment Scale-cognitive subscale (ADAS-cog), the clinical global impression of change scale (CGIC-plus scale), the Alzheimer's Disease Cooperative Study Activities of Daily Living Scale (ADCS-ADL), clinical global impression of change scale (CIBIC-plus scale); the Mini Mental State Exam (MMSE); the Neuropsychiatric Inventory (NPI); the Clinical Dementia Rating Scale (CDR); the Cambridge Neuropsychological Test Automated Battery (CANTAB) or the Sandoz Clinical Assessment-Geriatric (SCAG). See Folstein et al., J Psychiatric Res 12: 189-98, (1975); Robbins et al., Dementia 5: 266-81, (1994); Rey, L'examen clinique en psychologie, (1964); Kluger et al., J Geriatr Psychiatry Neurol 12:168-79, (1999). In addition, cognitive function may be measured using imaging techniques such as Positron Emission Tomography (PET), functional magnetic resonance imaging (fMRI), Single Photon Emission Computed Tomography (SPECT), or any other imaging technique that allows one to measure brain activity.

In animal model systems, cognitive function may be measured in various conventional ways known in the art, including using a Morris Water Maze (MWM), Barnes circular maze, elevated radial arm maze, T maze or any other mazes in which the animals use spatial information. Other tests known in the art may also be used to assess cognitive function, such as novel object recognition and odor recognition tasks.

Cognitive function may also be measured using imaging techniques such as Positron Emission Tomography (PET), functional magnetic resonance imaging (fMRI), Single Photon Emission Computed Tomography (SPECT), or any other imaging technique that allows one to measure brain function. In animals, cognitive function may also be measured with electrophysiological techniques.

“Age-related cognitive impairment” or “function” refers to cognitive function in aged subjects that is not as robust as that expected in an age-matched normal subject (i.e. subjects with mean scores for a given age in a cognitive test) or as that expected in young adult subjects. In some cases, cognitive function is reduced by about 5%, about 10%, about 30%, or more, compared to cognitive function expected in an age-matched normal subject. In some cases, cognitive function is as expected in an age-matched normal subject, but reduced by about 5%, about 10%, about 30%, about 50% or more, compared to cognitive function expected in a young adult subject. Age-related cognitive impairment may be associated with Mild Cognitive Impairment (MCI), Age-Associated Memory Impairment (AAMI), and Age-Related Cognitive Decline (ARCD).

“Mild Cognitive Impairment” or “MCI” refers to a condition characterized by isolated memory impairment unaccompanied other cognitive abnormalities and relatively normal functional abilities. One set of criteria for a clinical characterization of MCI specifies the following characteristics: (1) memory complaint (as reported by patient, informant, or physician), (2) normal activities of daily living (ADLs), (3) normal global cognitive function, (4) abnormal memory for age (defined as scoring more than 1.5 standard deviations below the mean for a given age), and (5) absence of indicators of dementia (as defined by DSM-IV guidelines; see also Petersen et al., Srch. Neurol. 56: 303-308 (1999); Petersen, “Mild cognitive impairment: Aging to Alzheimer's Disease.” Oxford University Press, N.Y. (2003)).

Diagnosis of MCI usually entails an objective assessment of cognitive impairment, which can be garnered through the use of well-established neuropsychological tests, including the Mini Mental State Examination (MMSE), the Cambridge Neuropsychological Test Automated Battery (CANTAB) and individual tests such as Rey Auditory Verbal Learning Test (AVLT), Logical

Memory Subtest of the revised Wechsler Memory Scale (WMS-R) and the New York University (NYU) Paragraph Recall Test. See Folstein et al., J Psychiatric Res 12: 189-98 (1975); Robbins et al., Dementia 5: 266-81 (1994); Kluger et al., J Geriatric Psychiatry Neurol 12:168-79 (1999).

“Age-Associated Memory Impairment (AAMI)” refers to a decline in memory due to aging. A patient may be considered to have AAMI if he or she is at least 50 years old and meets all of the following criteria: a) The patient has noticed a decline in memory performance, b) The patient performs worse on a standard test of memory compared to young adults, c) All other obvious causes of memory decline, except normal aging, have been ruled out (in other words, the memory decline cannot be attributed to other causes such as a recent heart attack or head injury, depression, adverse reactions to medication, Alzheimer's disease, etc.).

“Age Related Cognitive Decline (ARCD)” refers to declines in memory and cognitive abilities that are a normal consequence of aging in humans (e.g., Craik & Salthouse, 1992). This is also true in virtually all mammalian species. Age-Associated Memory Impairment refers to older persons with objective memory declines relative to their younger years, but cognitive functioning that is normal relative to their age peers (Crook et al., 1986). Age-Consistent Memory Decline, is a less pejorative label which emphasizes that these are normal developmental changes (Crook, 1993; Larrabee, 1996), are not pathophysiological (Smith et al., 1991), and rarely progress to overt dementia (Youngjohn & Crook, 1993). The DSM-IV (1994) has codified the diagnostic classification of ARCD.

### Description of Methods of the Invention

The methods of this invention comprise administration of a valproate or a pharmaceutically acceptable salt thereof, for example, sodium valproate, an antiepileptic medication for limbic seizures that modifies excitatory-inhibitory functions by increasing glutamate reuptake and GABA concentrations (Hassel et al., 2001; Loscher, 1999; Owens & Nemeroff, 2003), together with administration of an AChEI or a pharmaceutically acceptable salt thereof. In addition to epilepsy, sodium valproate has been prescribed for treatment of bipolar disorder, migraine, PTSD, and other neuropsychiatric disorders.

### Valproate

As used herein, the term “valproate” encompasses valproic acid, its analogs and derivatives, for example, compounds of the formula:

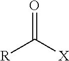

wherein, independently for each occurrence:

X is —OH, C₁₋₁₀ alkoxy, —O-alkali metal, —N(R¹)₂, —SH, or —S—C₁₋₁₀ alkyl;

R is a straight chain or branched C₁₋₃₀ alkyl; and

R¹ is H, C₁₋₁₀ alky, C₂₋₁₀ alkenyl, C₂₋₁₀ alkynyl, aryl, or aralkyl;

provided that R may be unsubstituted or substituted by one or more —OH, C₁₋₁₀ alkoxy, —N(R¹)₂, —SH, —S—C₁₋₁₀ alkyl, or aryl.
In certain embodiments of the invention, X is —OH, —O-alkali metal, —SH, or —NH₂; and R is CH[(CH₂)₂CH₃]₂.

Methods for making the valproates of this invention may be found in, for example, U.S. Pat. Nos.: 4,558,070; 4,595,695; 4,654,370; 4,895,873; 4,913,906; 5,017,613; 5,019,398; 5,049,586; 5,162,573; 5,440,023; 5,856,569; 6,131,106 and 6,610,326.

Pharmaceutically acceptable salts of the valproate may also be used in the methods and compositions of this invention. In some embodiments, the valproate of the invention is a combination of different valproates. Examples of valproate or its salt include, but are not limited to, 2-propylpentanoic acid, divalproex (mixture of valproic acid and valproate), sodium valproate, sodium divalproex, hydrogen bis(2-propyl pentanoate), calcium valproate, convulsofin, DEPAKENE™, DEPEKINE™, DEPAKOTE™, dipropyl acetate, sodium salt (2:1), semisodium valproate, Ergenyl, magnesium valproate, Vupral, valproamide, analogs of valproate and metabolites of valproate. In some embodiments, the valproic acid analog is 2-methylpent-2-enoic acid, or 2-methyl-2-n-propylpentanoic acid, or pent-4-enoic acid, or 2-ethylhexanoic acid, or valpromide, 2-propylpentanamide, or 2-n-alkylbut-3-ynoic acid (see Kostrouchova et al, 2007).

### AChEIs

As used herein, “acetylcholinesterase inhibitor” or “AChEI” has its ordinary meaning, and refers to an agent that blocks, suppresses, or reduces acetylcholinesterase activity. Pharmaceutically acceptable salts of an AChEI can also be used in the methods and compositions of this invention.

In some embodiments of this invention, the AChEI is donepezil or a salt, prodrug, or analog thereof, such as ARICEPT™. See U.S. Pat. Nos. 4,895,841, 5,985,864; 6,140,321, 6,245,911, 6,372,760. ARICEPT™ (donepezil hydrochloride) is a reversible inhibitor of the enzyme acetylcholinesterase, known chemically as (+)-2,3-dihydro-5,6-dimethoxy-2-[[1 [symbol] (phenylmethyl)-4-piperidinyl]methyl]-1H-inden-1-one hydrochloride.

Other exemplary AChEIs that are useful in the methods and compositions of this invention include tacrine (COGNEX™), rivastigmine (EXELON™), physostigmine (SYNAPTO™), galantharnine (REMINYL™), metrifonate (PROMEM™), quilostigmine, tolserine, thiatolserine, cymserine, thiacymserine, neostigmine, eseroline, ziftosilone, mestinon, huperzine A and icopezil. See U.S. Pat. Nos.: 4,895,841; 5,750,542; 5,574,046; 5,985,864; 6,140,321; 6,245,911; and 6,372,760.

Method of Treating Age-Related Cognitive Impairment with the Administration of a Valproate and an AChEI or Pharmaceutically Acceptable Salts Thereof

In some embodiments, the invention provides methods and compositions for improving age-related cognitive function and/or treating disorders involving age-related cognitive dysfunction, age-related cognitive impairment or the risk thereof in a subject in need thereof by administering a valproate or a pharmaceutically acceptable salt thereof in combination with an AChEI or a pharmaceutically acceptable salt thereof. In certain embodiments, the AChEI is donepezil. In certain embodiments, the valproate is sodium valproate. In certain embodiments, the age-dependent cognitive impairment is Mild Cognitive Impairment (MCI), Age-Related cognitive Decline (ARCD) or Age-Associated Memory Impairment (AAMI). The subject may be a human or other mammal such as a non-human primate, or rodent (e.g., rat). In one embodiment, the subject is a human patient.

When used clinically, donepezil shows a “cholinergic” side effect profile, and the dosage administered to patients is limited by such side effects. The use of the valproate and pharmaceutically acceptable salts thereof in combination with donepezil or other AChEIs and their pharmaceutically acceptable salts reduces the amount of donepezil or other AChEIs necessary for the treatment of disorders involving age-related cognitive dysfunction and other affective disorders and thus reduces the side effects caused by donepezil or other AChEIs without diminishing efficacy. Further, the efficacy of a combination of the valproate and donepezil or other AChEIs and pharmaceutically acceptable salts thereof exceeds the efficacy of either drug alone at its optimal dose and thus is a preferred treatment for diseases involving age-related cognitive dysfunction.

It will be appreciated that compounds and agents used in the compositions and methods of this invention preferably should readily penetrate the blood-brain barrier when peripherally administered. Compounds which cannot penetrate the blood-brain barrier, however, can still be effectively administered directly into the central nervous system, e.g., by an intraventricular route.

As used herein, administration of a valproate and an AChEI or pharmaceutically acceptable salts thereof “in combination” includes simultaneous administration and/or administration at different times, such as sequential administration. Simultaneous administration of the valproate and the AChEI or their pharmaceutically acceptable salts can optionally be combined with supplemental doses of the valproate and/or the AChEI and their salts. Simultaneous administration of drugs encompasses administration as co-formulation or, alternatively, as separate compositions.

In accordance with this invention, the valproate and the AChEI, and salts thereof, can be administered to a subject via any suitable route or routes. Most often, the drugs are administered orally; however, administration intravenously, subcutaneously, intra-arterially, intramuscularly, intraspinally, rectally, intrathoracically, intraperitoneally, intracentricularly, or transdermally, topically, or by inhalation is also contemplated. The agents can be administered orally, for example, in the form of tablets, troches, capsules, elixirs, suspensions, syrups, wafers, or the like, prepared by art recognized procedures. In certain embodiments, the administration is a slow, controll or extended release. The term “extended release” is widely recognized in the art of pharmaceutical sciences and is used herein to refer to a controlled release of an active compound or agent from a dosage form to an environment over (throughout or during) an extended period of time, e.g. greater than or equal to one hour. An extended release dosage form will release drug at substantially constant rate over an extended period of time or a substantially constant amount of drug will be released incrementally over an extended period of time. The term “extended release” used herein includes the terms “controlled release”, “prolonged release”, “sustained release”, or “slow release”, as these terms are used in the pharmaceutical sciences. In some embodiments, the extended release dosage is administered in the form of a patch or a pump.

When a solid carrier is used for administration, the preparation may be in a tablet, placed in a hard gelatine capsule in powder or pellet form, or it may be in the form of a troche or lozenge. If a liquid carrier is used, the preparation may be in the forms of a syrup, emulsion, soft gelatine capsule or sterile injectable liquid such as an aqueous or non-aqueous liquid suspension or solution.

Dosage schedules of the agents and compositions according to the methods of the invention will vary according to the particular compound or compositions selected, the route of administration, the nature of the condition being treated, the age and condition of the patient, the course or stage of treatment, and will ultimately be at the discretion of the attending physician. It will be understood that the amount of the valproate and the AChEI and their pharmaceutically acceptable salts administered will be amounts effective to produce a desired biological effect, such as beneficial results, including clinical results (e.g., an amount that increases GABAergic activity, reduces excitatory neurotransmission, and blocks, suppresses, or reduces acetylcholinesterase activity, and/or amounts that in combination result in an improvement in cognitive function). It will be understood that an effective amount can be administered in more than one dose and over a course of treatment.

In certain embodiments of the invention, the valproate or a pharmaceutically acceptable salt thereof is administered at a daily dose such that the subject maintains a blood total valproate level of 0.1 to 20 μg/ml plasma. In some embodiments, the valproate or a pharmaceutically acceptable salt thereof is administered at a daily dose of less than 1500 mg, less than 1000 mg, less than 500 mg, less than 250 mg, less than 125 mg, less than 75 mg, less than 25 mg, less than 10 mg, or less than 5 mg. For repeated administrations over several days or weeks or longer, depending on the condition, the treatment is sustained until a sufficient level of cognitive function is achieved. In some embodiments, a subtherapeutic amount of the valproate or its pharmacologically acceptable salt is administered.

The AChEI and its salts may be administered at a dosage level up to conventional dosage levels. Suitable dosage levels will depend upon the specific AChEI or its salt that is chosen, but typically suitable levels will be about 5 to 10 mg, or less. The AChEI or its salt may be administered on a regimen of up to 2 times per day, 1 time per day, or it may be administered less often. For ARICEPT™, a typical daily dosage when administered alone is about 5 to 20 mg.

In certain embodiments of the invention, the amount of ARICEPT™ administered in combination with a valproate or a salt thereof is about 0.1 to 10 mg. In some embodiments, the amount of ARICEPT™ administered in combination with the valproate or its salt is a subtherapeutic amount. In some embodiments, the amount of ARICEPT™ administered in combination with the valproate or a salt thereof is less than 10 mg daily, less than 5 mg daily, less than 1 mg daily, less than 0.5 mg daily, or less than 0.1 mg daily.

The AChEI or a salt thereof may be administered at dosage levels distinct from conventional levels when provided in combination with a valproate or a salt thereof, due to a valproate-dependent increase in the AChEI's therapeutic index. In some embodiments, the increase in the AChEI's therapeutic index due to the combination with a valproate or a salt thereof is greater than the therapeutic index of the AChEI alone by at least about 1.5× or 2.0× or 2.5× or 3.0× or 3.5× or 4.0× or 4.5× or 5.0× or 5.5× or 6.0× or 6.5× or 7.0× or 7.5× or 8.0× or 8.5× or 9.0× or 9.5× or 10×, or greater than about 10×. In some embodiments, combinations of an AChEI or it salt with valproate or its salt reduces the dosage of the AChEI required for its therapeutic effect. In some embodiments, the amount of the AChEI administered in combination with the valproate or a salt thereof is about 0.1 to 10 mg. In some embodiments, the amount of the AChEI administered in combination with the valproate or its salt is a subtherapeutic amount. In some embodiments, the amount of the AChEI administered in combination with the valproate or its salt is less than 10 mg daily, less than 5 mg daily, less than 1 mg daily, less than 0.5 mg daily, or less that 0.1 mg daily.

The frequency of administration of the composition of this invention may be adjusted over the course of the treatment, based on the judgment of the administering physician. It will be clear that the valproate and the AChEI and their salts can be administered at different dosing frequencies or intervals. For example, valproate or its salt can be administered daily (including multiple doses per day) or less frequently. An AChEI or its salt can be administered daily (including multiple doses per day) or less frequently. In some embodiments, sustained continuous release formulations of a valproate and an AChEI or its salt may be desired. Various formulations and devices for achieving sustained release are known in the art.

As described above, some AChEIs (such as donepezil) and their salts can cause cholinergic side effects. The use of a combination of a valproate and an AChEI and their salts may reduce the amount of the AChEI necessary for treatment of disorders involving age-related cognitive dysfunction and other affective disorders and may thus reduce the side effects caused by the AChEIs. In particular, the combination of a valproate with a reduced amount of AChEI or its salt may reduce the cholinergic side effects without negatively impacting efficacy. Accordingly, in some embodiments, a subtherapeutic amount of AChEI is administered.

In some embodiments, enough of the valproate or its salt is administered so as to allow the dose of the AChEI or its salt (e.g., a dose required to effect a degree of cognitive function improvement or treat age-associated cognitive impairment) to be reduced by at least about 20%, at least about 30%, at least about 40%, or at least about 50%, at least about 60%, at least about 70%, at least about 80%, at least about 90% or more from to the dose of AChEI or its salt normally used when administered alone (i.e., individually and not in combination with other therapeutic agents or compounds). The reduction may be reflected in terms of amount administered at a given administration and/or amount administered over a given period of time (reduced frequency).

Compositions of this Invention

In one aspect, the invention provides compositions containing a valproate and an AChEI and their salts. In some embodiments, the valproate and the AChEI or their salts may be present in a single dosage unit (e.g., combined together in one capsule, tablet, powder, or liquid, etc.). In some embodiments, the AChEI in the composition is donepezil. In some embodiments, the composition includes sodium valproate as the valproate, and includes donepezil as the AChEI. The composition described herein can contain more than one valproate and/or more than one AChEI or their salts.

The compositions described herein can further contain pharmaceutically acceptable excipient(s) and may contain other agents that serve to enhance and/or complement the effectiveness of the valproate and/or the AChEI or their salts. The compositions may also contain additional agents known to be useful for treating cognitive function disorder.

The composition in the present invention may be in solid dosage forms such as capsules, tablets, dragrees, pills, lozenges, powders and granule. Where appropriate, they may be prepared with coatings such as enteric coatings or they may be formulated so as to provide controlled releases of one or more active ingredient such as sustained or prolonged release according to methods well known in the art. In certain embodiments, the composition is in form of a slow, controll or extended release. The term “extended release” is widely recognized in the art of pharmaceutical sciences and is used herein to refer to a controlled release of an active compound or agent from a dosage form to an environment over (throughout or during) an extended period of time, e.g. greater than or equal to one hour. An extended release dosage form will release drug at substantially constant rate over an extended period of time or a substantially constant amount of drug will be released incrementally over an extended period of time. The term “extended release” used herein includes the terms “controlled release”, “prolonged release”, “sustained release”, or “slow release”, as these terms are used in the pharmaceutical sciences. In some embodiments, the extended release dosage is administered in the form of a patch or a pump. The composition may also be in liquid dosage forms including solutions, emulsions, suspensions, syrups and elixirs.

The compositions may be specifically formulated for administration by any suitable route as described herein and known in the art. Compositions for parental administration include sterile aqueous and nonaqueous injectable solutions, dispersions, suspensions or emulsions as well as sterile powders to be reconstituted in sterile injectable solutions or dispersions prior to use. Compositions for intraoral and oral delivery (including sublingual and buccal administration, e.g. Danckwerts et al, and oral) include but are not limited to bioadhesive polymers, tablets, patches, liquids and semisolids (see e.g Smart et al). Compositions for respiratory delivery (pulmonary and nasal delivery) include but are not limited to a variety of pressurized metered dose inhalers, dry powder inhalers, nebulizers, aqueous mist inhalers, drops, solutions, suspensions, sprays, powders, gels, ointments, and specialized systems such as liposomes and microspheres (see e.g. Owens et al, “Alternative Routes of Insulin Delivery” and Martini et al). Compositions for transdermal delivery include but are not limited to colloids, patches and microemulsions. Other suitable administration forms for the above and other include depot injectable formulations, suppositories, sprays, ointments, cremes, gels, inhalants, dermal patches, implants etc.

Therapeutic formulations can be prepared by methods well known in the art of pharmacy, see, e.g., Goodman et al., 2001; Ansel, et al., 2004; Stoklosa et al., 2001; and Bustamante, et al., 1993.

In certain embodiments of the invention, a composition containing a valproate and an AChEI and their salts comprises an amount of the valproate or its salt is such that the subject maintains a blood total valproate level of 0.1 to 20 μg/ml plasma. In some embodiments, the amount of the valproate or its salt in the composition is less than 1500 mg, less than 1000 mg, less than 500 mg, less than 250 mg, less than 125 mg, less than 75 mg, less than 25 mg, less than 10 mg, or less than 5 mg. In some embodiments, the amount of the AChEI or its salt in the composition is about 0.1 to 10 mg. In some embodiments, the amount of the AChEI or its salt in the composition is less than 10 mg, less than 5 mg, less than 2 mg, less than 1 mg, less than 0.5 mg, or less than 0.1 mg.

It will be understood by one of ordinary skill in the art that the compositions and methods described herein may be adapted and modified as is appropriate for the application being addressed and that the compositions and methods described herein may be employed in other suitable applications, and that such other additions and modifications will not depart from the scope hereof.

This invention will be better understood from the Experimental Details which follow. However, one skilled in the art will readily appreciate that the specific methods and results discussed are merely illustrative of the invention as described more fully in the embodiments which follow thereafter.

### Examples

Introduction and Models of Age-Related Cognitive Impairment

A variety of conditions characterized by cognitive impairment (e.g., Age Associated Memory Impairment [AAMI], Mild Cognitive Impairment [MCI] and Age-Related Cognitive Decline [ARCD]) are believed to be related to aging. Animal models serve as an important resource for developing and evaluating treatments for such age-related cognitive impairments. Features that characterize age-related cognitive impairment in animal models typically extend to age-related cognitive impairment in humans. Efficacy in such animal models is, thus, predictive of efficacy in humans.

Of available models, a Long-Evans rat model of cognitive impairment is particularly well suited for distinguishing the difference between cognitive impairment related to illness and that related to aging. Indeed, extensive behavioral characterization has identified a naturally occurring form of cognitive impairment in an outbred strain of aged Long-Evans rats (Charles River Laboratories; Gallagher et al., Behav. Neurosci. 107:618-626, (1993)). In a behavioral assessment with the Morris Water Maze (MWM), rats learn and remember the location of an escape platform guided by a configuration of spatial cues surrounding the maze. The cognitive basis of performance between young rats (6 months of age), and aged rats were (26-27 months of age) are tested in probe trials using measures of the animal's spatial bias in searching for the location of the escape platform. Aged rats in the study population have no difficulty swimming to a visible platform, but an age-dependent impairment is detected when the platform is camouflaged, requiring the use of spatial information. Performance for individual aged rats in the outbred Long-Evans strain varies greatly. For example, a proportion of those rats perform on a par with young adults. However, approximately 40-50% fall outside the range of young performance. This variability among aged rats reflects reliable individual differences. Thus, within the aged population some animals are cognitively impaired and designated aged-impaired (AI) and other animals are not impaired and are designated aged-unimpaired (AU). See, e.g., Colombo et al., Proc. Natl. Acad. Sci. 94: 14195-14199, (1997); Gallagher and Burwell, Neurobiol. Aging 10: 691-708, (1989); Rapp and Gallagher, Proc. Natl. Acad. Sci. 93: 9926-9930, (1996); Nicolle et al., Neuroscience 74: 741-756, (1996); and Nicolle et al., J. Neurosci. 19: 9604-9610, (1999), Gallagher et al. Behav. Neurosci. 107:618-626, (1993).

We used this rat model described above to identify individual AI and AU rats. We then conducted histological experiments on them, as well as tested their performance in further MVM tasks while administering various pharmacological treatments.

### Example 1

### Effect of Valproate Treatment in Muscarinic Coupling

Membrane preparation: Rats were decapitated and the hippocampus was dissected on ice then frozen at −80° C. All the tissue was stored in −80° C. and processed for GTP-Eu binding. For the GTP-Eu binding assay, membranes were prepared from frozen tissue by gentle homogenization with a Dounce homogenizer in five volumes of ice-cold 50 mM Tris buffer, pH 7.4, containing 0.2 mM EGTA and protease inhibitors. After centrifuging at 40,000×g at 4° C. for 10 min, the pellets were resuspended in 30 ml of ice-cold Tris buffer. The steps described above were repeated twice, and then the final pellets were resuspended in assay buffer (50 mM Tris, 0.2 mM EGTA, 5 mM MgCl₂, and 100 mM NaCl, pH 7.4) (Zhang et al 2007).

Muscarinic receptor-stimulated GTP-Eu binding assay. GTP-Eu binding was assessed using time-resolved fluorescence (Wallac 1420, Perkin-Elmer). The membranes and all the reagents were diluted with assay buffer. Samples were added into a 96-well filter plate in a counter-balanced manner based upon age and behavioral performance, although the experimenter was blind to the identification of the groups at the time of the assay. The samples were incubated at 37° C. for 40 min then washed four times with GTP washing buffer from the kit. The integration of GTP-Eu into the membrane was measured by excitation of the samples at 340 nm and reading the emission at 615 nm using time-resolved fluorescence (Zhang et al 2007, Nicolle et al 1999).

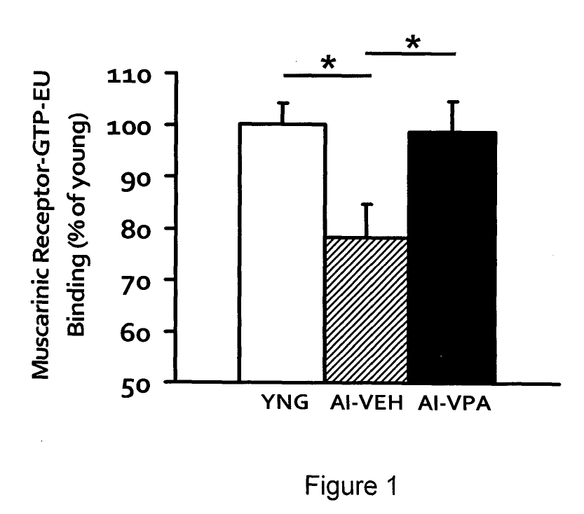

Results: Data from the binding assay are presented as a percentage of the muscarinic receptor-stimulated GTP-Eu binding in a young rat. Data indicates that a low dose of chronic valproate administration was found to effectively restore post-synaptic muscarinic coupling in the hippocampus of memory impaired aged rats (FIG. 1).

### Example 2

### Behavioral Assessment of Sodium Valproate (“VPA”) Treatment in a 6 Hour-Delay Memory Task

Aged rats that demonstrated impaired memory performance in a standardized assessment of spatial cognition (Gallagher et al., 1993), i.e., AI rats, were selected for the drug intervention studies. AI Rats were treated with sodium valproate (“VPA”) chronically and subcutaneously at a rate of 100 mg/kg/day via implanted osmotic minipump, and then trained and tested in a new water maze environment. The water maze used here was housed in a different building and was surrounded by black curtains with a novel set of white patterns. The training protocol used was based on a modified water maze task known to be highly hippocampal-dependent (de Hoz et al., 2005; Steele & Morris, 1999). Unlike the traditional water maze protocol (such as that used in the background assessment, described above) wherein the escape platform location remained constant throughout training, the escape platform location in this spatial memory version of the task varied from day to day.

The test was comprised of a training phase followed by a memory retention test. Each day, during the training phase, rats were given six trials to locate the submerged escape platform. On each trial, a rat was released in the maze from one of four equally spaced starting positions around the perimeter of the pool. The starting position varied from trial to trial. If the rat did not locate the escape platform within 60 s on any trial, the experimenter guided and placed the rat on the platform, where it remained for 20 s. The rat was then removed from the platform and placed in a holding cage for another 40 s before the next trial. The length of the path the rat took while swimming in the maze was measured in each trial. At the end of the 6 trials of the training session, the rats were returned to their home cages in the colony room for a 6-hr waiting period, in preparation for the subsequent memory retention test.

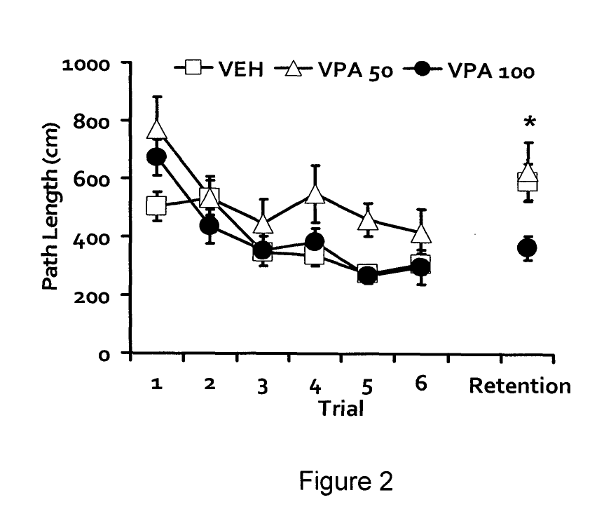

After the 6-hr waiting period, the rats were returned to the water maze for a test of spatial memory with the submerged platform located in the same position as in the training trials. Each rat was given 60 s to locate the platform. Rats were trained and tested in this manner for five consecutive days. Performances on the last three days were averaged for analysis. Results are shown in FIG. 2. Memory-impaired aged rats treated with sodium valproate or saline performed at a comparable level at the end of the training phase (Trials 1-6), but those treated with a higher dose of VPA (VPA 100=100 mg/kg/day) showed less forgetting (i.e., memory savings or retention) after a 6-hr delay compared to their counterparts treated with either saline (VEH) or a lower dose of the drug (VPA 50=50 mg/kg/day). The chronic subcutaneous administration of 100 mg/kg/day VPA results in a blood total VPA level of 10 μg/ml plasma (10 μg/ml total VPA).

### Example 3

### Behavioral Assessment of VPA, Donezepil and Combination Treatments in a 24 Hour-Delay Memory Task

Age-Impaired (AI) Long-Evans rats (as characterized above) were tested for their memory of new spatial information in the MWM, under different drug/control treatment conditions: vehicle control, donezepil only, VPA only, and combination of VPA and donezepil. Donezepil was administered at a range of doses (0.5, 1.0 or 2.0 mg/kg) 30 minutes to 1 hour prior to training trials. VPA was administered chronically and subcutaneously at a rate of 100 mg/kg/day via implanted osmotic minipump.

AI rats were given six training trials per training day with a 60-sec inter-trial interval between each training trial for two consecutive days. On each training trial, the rat was released in the maze from one of four equally spaced starting positions around the perimeter of the pool. If the rat did not locate the escape platform within 90 sec on any trial, the experimenter guided the rat to the platform, where it remained for 30 sec. 30 minutes to 1 hour prior to all the training trials on each training day, AI rats were pretreated with one of four drug conditions: 1) vehicle control (0.9% saline solution); 2) 0.5 mg/kg donpepzil; and 3) 1.0 mg/kg donpepzil; and 4) 2.0 mg/kg donpepzil through intraperitoneal (i.p.) injection.

The same AI rats were used for the entire set of experiments so that each treatment condition was tested on each of the rats. Therefore, to counterbalance any potential bias, both the location of the escape platform and the spatial cues surrounding the water maze were different in the four treatment conditions. In other words, using one set of locations and spatial cues, two rats were treated with saline control solution, two with 0.5 mg/kg donpepzil, two with 1.0 mg/kg donpepzil and two with 2.0 mg/kg donpepzil. These groups were cycled through 3 more sets of locations and spatial cues, with each group of two receiving a different treatment each time, so that at the end, each treatment condition was tested in eight rats.

After the completion of the twelve training trials (over the two days), the rats were returned to their home cages in the colony room for a 24-hr waiting period, in preparation for the subsequent memory retention test. After the waiting period, the rat was given one testing trial (the “retention trial”), which was the same MWM task as the training trials, but with the escape platform removed.

For the retention trial, the MWM circular pool was divided into 4 quadrants. The particular quadrant where the escape platform was placed in the training trials is referred to as the “target quadrant”. The particular region where the platform was located in the training trials is referred to as the “target annulus”. In the retention trial, the time the AI rats spent swimming in the target quadrant was measured and further plotted as a percentage of total swimming time. The time the AI rats spend in the target annulus was also measured.

To study the effect of vaproate treatment and combination VPA/donepezil treatment, the above experiment was repeated with AI rats implanted with an osmotic minopump administering VPA chronically and subcutaneously at a rate of 100 mg/kg/day.

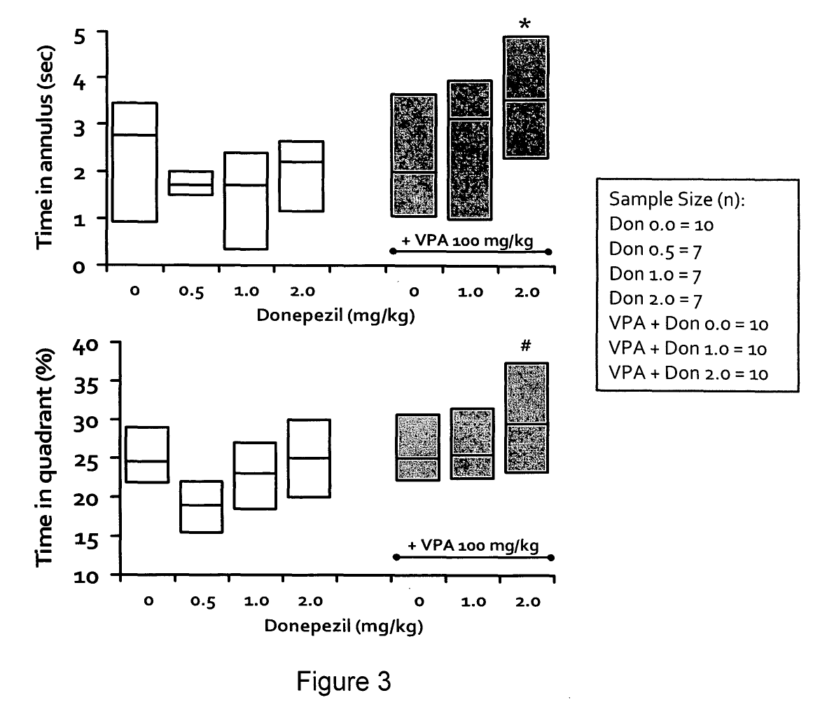

In the retention trial, whose results are depicted in FIG. 3, the time the AI rats spent in the target quadrant was approximately 25%, which is a performance equivalent to them having no memory of the platform location. This performance did not significantly improve in the group treated with donezepil at any of the doses tested (0.5, 1.0, and 2.0 mg/kg). Further, no improvement was seen in the performance of AI rats treated with VPA only. However, combined administration of 2.0 mg/kg donezepil with 100 mg/kg/day VPA resulted in improved performance as compared to vehicle-treated controls, as indicated by an increase in the time spent in the target annulus or target quadrant (see FIG. 3).

While 100 mg/kg/day VPA-only treatment improved the performance of AI rats in the memory retention test after a 6-hour waiting period after training (see Example 2), there was no improvement seen in the more difficult memory retention test with a 24-hour waiting period. Donezepil-treatment also had no effect in the performance of AI rats in the 24-hour retention test at any dosage tested, even at the maximum tested dose of 2.0 mg/kg. However, when these subtherapeutic doses of VPA (100 mg./kg/day) and donezepil (2.0 mg/kg) were combined, there was an unexpected and synergistic improvement in the memory retention of AI rats.

### Example 4

### Behavioral Assessment of VPA, Donezepil and Combination Treatments in a 8 or 12-Arm Radial Maze Memory Test

The effect of administration of a combination of VPA with donepezil on the spatial memory of rats can be measured in a 1 or 8 hour retention test on a twelve-arm radial maze. Behavioral testing is conducted by an experimenter blinded to drug treatment. 12 Long-Evans rats trained to use a win-shift strategy are given an information trial. During the information trial, 5 of the 12 arms of the 12 arm maze are blocked so that rats are not able to consume food from those blocked arms but can obtain food from each of the 7 open arms. After this session, rats are moved to their home cage and placed back in the animal holding room. Eight hours later (memory test) rats are reintroduced into the maze with all arms open and only the previously blocked arms are baited. Memory for the 7 arms in the information session is demonstrated when rat visits only the previously blocked arms on the memory test. A retroactive memory error is made when the rat enters an arm that was open on the information trial. In the case of an 8 arm maze, 3 of the 8 arms are blocked during the information trial.

A within-subject design is employed to examine drug treatments as a single dose. 12 rats are used in the experiment. The rats are treated with VPA, donepezil, or combination of VPA and donepezil, in a range of concentrations. VPA is administered chronically via osmotic minipump (up to 100 mg/kg/day). Donepezil (up to 2 mg/kg) is administered sixty minutes prior to each information session. Physiological saline (NaCl) is used as vehicle. Treatment is counterbalanced such that the rats receive a different order of treatment conditions across days. In both the 1 hour and 8 hour delay memory tasks, the dose of donepezil at which improvement in the memory task is observed is reduced when the rats are treated in combination with VPA.

1. A method of treating age-related cognitive impairment in a subject in need or at risk thereof, the method comprising the step of administering to said subject a therapeutically effective amount of an Acetylcholinesterase Inhibitor (AChEI) and a valproate or their pharmaceutically acceptable salts.

2. The method of claim 1, wherein the valproate and/or the AChEI or their pharmaceutically acceptable salts are administered at a dose that is subtherapeutic when administered individually.

3. The method of claim 1, wherein the age-related cognitive impairment is Mild Cognitive Impairment (MCI).

4. The method of claim 1, wherein the valproate or a pharmaceutically acceptable salt thereof administered at a daily dose such that the subject maintains a blood total valproate level of 0.1 to 20 μg/ml plasma.

5. The method of claim 1, wherein the subject is a human patient.

6. The method of claim 1, wherein the AChEI is donepezil, tacrine, rivatigmine, physostigmine or metrifonate or their pharmaceutically acceptable salts.

7. The method of claim 6, wherein the AChEI is donepezil or a pharmaceutically acceptable salt thereof.

8. The method of claim 1, wherein the AChEI or a pharmaceutically acceptable salt thereof is administered at a daily dose of 0.1 mg to 10 mg.

9. The method of claim 1 wherein the valproate and the AChEI or their pharmaceutically acceptable salts are administered simultaneously.

10. The method of claim 10, wherein the valproate and the AChEI or their pharmaceutically acceptable salts are administered in a single formulation.

11. The method of claims 1, wherein the valproate and the AChEI or their pharmaceutically acceptable salts are administered sequentially.

12. The method of claim 2, wherein the the AChEI is administered at a daily dose of less than 10 mg, less than 5 mg, less than 2 mg, less than 1 mg, less than 0.5 mg, or less than 0.1 mg.

13. The method of claim 2, wherein the the valproate is administered at a daily dose of less than 1500 mg, less than 1000 mg, less than 500 mg, less than 250 mg, less than 125 mg, less than 75 mg, less than 25 mg, less than 10 mg, or less than 5 mg.

14. A method of increasing the therapeutic index of an AChEI, comprising administering the valproate in combination with the AChEI to a subject.

15. The method of claim 14, wherein the increase in the therapeutic index of the the AChEI in combination with the valproate is greater than the therapeutic index of the AChEI alone by at least about 1.5× or 2.0× or 2.5× or 3.0× or 3.5× or 4.0× or 4.5× or 5.0× or 5.5× or 6.0× or 6.5× or 7.0× or 7.5× or 8.0× or 8.5× or 9.0× or 9.5× or 10×, or greater than about 10×.

16. A pharmaceutical composition comprising an AChEI and a valproate or their pharmaceutically acceptable salts.

17. The composition of claim 16, wherein the composition is in a solid form.

18. The composition of claim 16, wherein the composition is in a liquid form.

19. The composition of claim 16, wherein the composition is in a unit dosage form.

20. The composition of claim 16, wherein the valproate or a pharmaceutically acceptable salt thereof in the composition is in an amount such that the subject maintains a blood total valproate level of 0.1 to 20 μg/ml plasma.

21. The composition of claim 16, wherein the AChEI is donepezil, tacrine, rivatigmine, physostigmine or metrifonate or pharmaceutically acceptable salts thereof.

22. The composition of claim 21, wherein the AChEI is donepezil or a pharmaceutically acceptable salt thereof.

23. The composition of claim 16, wherein the AChEI or a pharmaceutically acceptable salt thereof is present in an amount of 0.1 mg to 10 mg.

24. The composition of claim 16, wherein the AChEI or a pharmaceutically acceptable salt thereof is present in an amount of less than 10 mg, less than 5 mg, less than 2 mg, less than 1 mg, or less than 0.5 mg.

25. The composition of claim 16, wherein the valproate or a pharmaceutically acceptable salt thereof is present in an amount less than 1500 mg, less than 1000 mg, less than 500 mg, less than 250 mg, less than 125 mg, less than 75 mg, less than 25 mg, less than 10 mg, or less than 5 mg.
